(19) United States
(12) Patent Application Publication
(10) Pub. No.: US 2013/0190730 A1
(43) Pub. Date: Jul. 25, 2013

(54) CATHETER AND CATHETER SYSTEM

(71) Applicant: VASCOMED GMBH, Binzen (DE)

(72) Inventors: Stefan Knorr, Berlin (DE); Ingo Weiss, Berlin (DE); Stephan Fandrey, Affoltern am Albis (CH); Wolfgang Geistert, Rheinfelden (DE)

(73) Assignee: VASCOMED GMBH, Binzen (DE)

(21) Appl. No.: 13/741,956

(22) Filed: Jan. 15, 2013

Related U.S. Application Data
(60) Provisional application No. 61/588,192, filed on Jan. 19, 2012.

(51) Int. Cl. A61M 25/10

(52) U.S. Cl. CPC A61M 25/10
USPC 604/523; 604/96.01

(57) ABSTRACT

A catheter comprising an elongated catheter body which has a distal end and a proximal end in respect of an operating position, wherein the catheter body comprises an inner tube and an outer tube which is displaceable relative thereto, and a deformation body is disposed on the distal end which is connected in the distal region thereof to the distal end of the inner tube, and in the proximal region thereof to the distal end of the outer tube.

### CROSS-REFERENCE TO RELATED APPLICATIONS

This patent application claims the benefit of co-pending U.S. Provisional Patent Application No. 61/588,192, filed on Jan. 19, 2012, which is hereby incorporated by reference in its entirety.

### TECHNICAL FIELD

The present invention relates generally to a catheter comprising an elongated catheter body which has a distal end and a proximal end in respect of an operating position. It furthermore relates to a catheter system comprising such a catheter and a measuring device attached thereto.

### BACKGROUND

In the field of medicine, a large number of catheters having various designs or catheters-like devices (e.g., electrode leads) are known and are used on a large scale. They are used in part by experienced specialists, but also in part by physicians or medical personnel who do not have special knowledge or experience. Regardless, harm and/or injury to the patient must be reliably ruled out.

Known catheters comprising a plastic or metal tip have a perforation risk. To minimize the surface pressure and, therefore, the risk of perforation, compromises must be struck in respect of stiffness of the catheter shaft and the catheter tip. Under certain circumstances, these compromises limit the maneuverability and positional stability of the catheter.

A problem addressed by the present invention is therefore that of providing an improved catheter which can be used even by less experienced personnel without risk of harm and/or injury to the patient, yet is designed—in respect of flexibility in particular—such that the requirements for clinical application are still met.

The present invention is directed toward overcoming one or more of the above-identified problems.

### SUMMARY

An object of the present invention is solved by a catheter having the features of claim 1. The present invention furthermore provides a catheter device having the features of claim 17.

The present invention is based on the idea of modifying known catheter designs by providing a special perforation protection element. It also involves the idea of providing a deformation body on the distal end of the catheter. Finally, the present invention involves the idea of an (at least) two-layer design of the catheter body, wherein the catheter has an outer tube and an inner tube which are interconnected by a deformation body, wherein the inner tube is connected to the distal end of the deformation body, and the outer tube is connected to the proximal end of the deformation body. When an axial force acts on the catheter tip, the deformation body deforms and the inner tube slides in a telescopic manner into the outer tube.

An embodiment in which the deformation body has a larger diameter than the actual catheter tip has an advantage that the supporting surface of the catheter on the tissue and, therefore, the surface pressure is additionally reduced.

In a further embodiment, the deformation body is in the form of a balloon with a fluid filling. According to one embodiment of this variant, the balloon has a fluid connection—via a fluid channel in the catheter body—with one or more fluid connectors on the proximal end of the catheter. More specifically, this variant can be designed such that the balloon is filled with an elastically compressible or incompressible fluid.

Using a balloon as the deformation body offers an advantage that it can be inserted in the deflated state, and so the insertion cross-section thereof need not be much greater than that of the catheter shaft. In addition, when a balloon is used, the pressing surface and/or the pressure can be deduced via the pressure change that occurs when pressed against the tissue, and via the measurement thereof.

In another variant, the deformation body is made of an elastically compressible material.

In each of the aforementioned embodiments, but also as an independently functional embodiment, the deformation body can comprise a substantially axially acting compression spring device, in particular, for example, a metal or plastic spring.

In a further embodiment, the deformation body comprises a flexibly yielding sleeve or jacket layer.

In respect of the mechanical properties that are essential to the function of the catheter, the variants are therefore as follows. The deformation body:
can be a balloon that is filled with a medium, the pressure of which determines the hardness and, therefore, the spring force of the balloon, or
can be made of a soft plastic or a foam that determines the spring force by way of the elastic properties thereof, or
can also be a common spring that determines the spring force by way of the spring constant thereof.

When the inventive catheter is used, the deformation body undergoes a pressure change when pressed against the wall of a vessel or hollow organ.

The pressure change can be converted distally or proximally. The pressure can be measured as follows:
Transmit the pressure in the hydraulic system from the distal end to the proximal end of the catheter. The pressure information is converted on the proximal end (e.g., in the ablation device or in the pump).
The pressure is converted in the distal end of the catheter and is transmitted electrically or optically to the proximal end. Various methods can be used to measure pressure, which will be apparent to one of ordinary skill in the art.
The optical properties of the medium in the balloon can be measured in order to deduce the deformation of the deformation body and, therefore, the pressure of the catheter on the tissue.
If a soft plastic or a foam is used as the deformation body, the pressure change that occurs due to pressing against the tissue can result in a change in the electrical properties of the deformation body (e.g., resistance, capacitance, etc.), which can also be measured and utilized to determine the pressing surface and/or pressure.

In a further embodiment, the deformation body has a multiple-component design and is formed of a plurality of chambers or partial bodies, wherein the chambers or partial bodies have different deformation behavior and/or separate measuring means or connectors for detecting a specific pressure applied thereto.

In particular, each of the chambers has a fluid connection via a fluid channel in the catheter body to one or more fluid connectors on the proximal end of the catheter, or the partial bodies comprise optically and/or electrically acting measuring means or connections.

In a further embodiment, the inventive catheter is in the form of an electrode lead comprising at least one electrode disposed on the deformation body or on the distal end of the inner tube. In the latter case, the electrode is enclosed by the deformation body. The electrode, or the at least one electrode, is elastically deformable, in particular, being made of, for example, a conductive plastic in particular.

Further features, aspects, objects, advantages, and possible applications of the present invention will become apparent from a study of the exemplary embodiments and examples described below, in combination with the figures, and the appended claims.

### DESCRIPTION OF THE DRAWINGS

Advantages and useful features of the present invention will also become apparent from the basic description that follows of embodiments, with reference to the figures. In the figures:

FIGS. 1A-1D show schematic representations (side views or cross-sectional views) of an embodiment of the catheter according to the present invention, in the starting state and in the deformed state of the catheter end;

FIGS. 2A-2B show schematic, perspective representations of a further embodiment of the inventive catheter;

FIGS. 3A-3B show schematic, perspective representations of a further embodiment of the inventive catheter;

FIGS. 4A-4B show a perspective depiction and cross-sectional view of a further embodiment of the catheter according to the present invention;

FIG. 5 shows a schematic depiction of an embodiment of the proposed inventive catheter as a bipolar electrode lead; and

FIGS. 6A-6B show schematic diagrams of embodiments of the catheter system according to the present invention.

### DETAILED DESCRIPTION

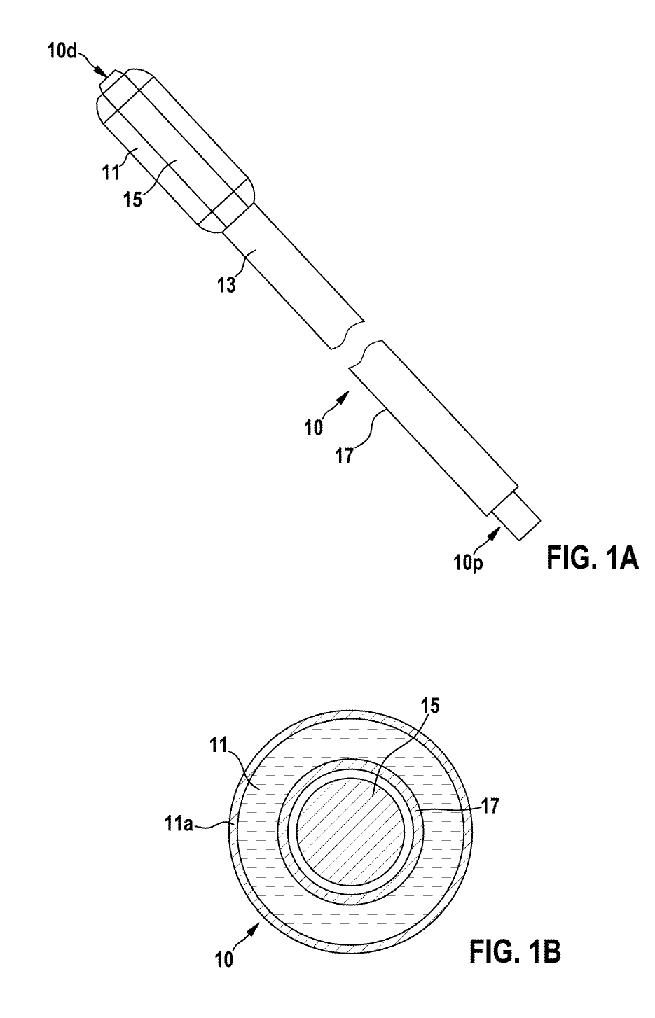

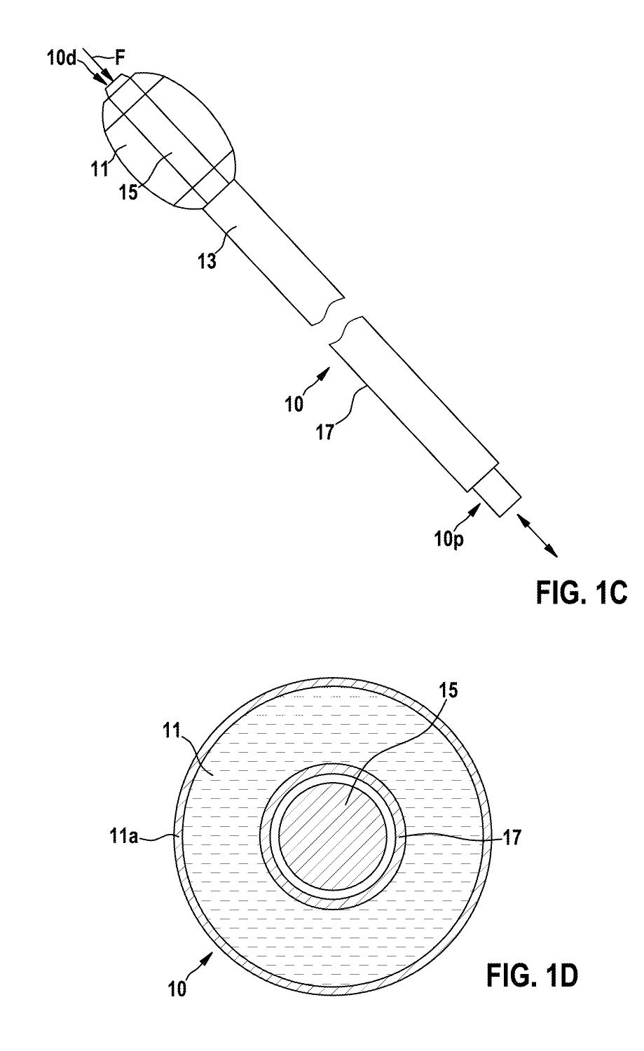

FIGS. 1A-1D show schematic illustrations of a catheter 10 having a distal end 10d and a proximal end 10p, wherein a deformation body 11 is mounted on the distal end 10d. In the embodiment shown, the deformation body 11 is a balloon having a flexibly yielding sleeve 1 la which deforms in bulbous manner when the distal catheter end 10d contacts the wall of a vessel or organ. Balloon 11 is filled with a suitable fluid which is, for example, a pressurized gas, but basically can also be a fluid or a quantity of solid particles (which are compressible in particular).

A catheter body 13 of catheter 10 comprises an inner tube 15 and, proximally displaceable relative thereto, an outer tube 17. The distal end of balloon 11 is attached to the distal end of the inner tube 15, and the proximal end of balloon 11 is attached to the distal end of the outer tube 17. Since the inner tube 15 and the outer tube 17 are displaceable relative to one another, as shown in FIG. 1C, the action of a compressive force “F” on the distal end 10d of the catheter 10 causes deformation of balloon 11 and a displacement of inner tube 15 relative to outer tube 17 in the proximal direction. This displacement can be detected and evaluated; the use of the proposed catheter for measurement purposes is described with reference to the embodiments below.

If a fluid or a gas (e.g., saline solution or carbon dioxide) is used as the inflation medium, it can be supplied via one of the tubes—preferably via outer tube 17, according to a current perspective—and the deformation behavior of the distal catheter end can be set by adjustment of the pressure. If the balloon 11 is expandable, the size thereof can also be adjusted as desired via the fluid pressure.

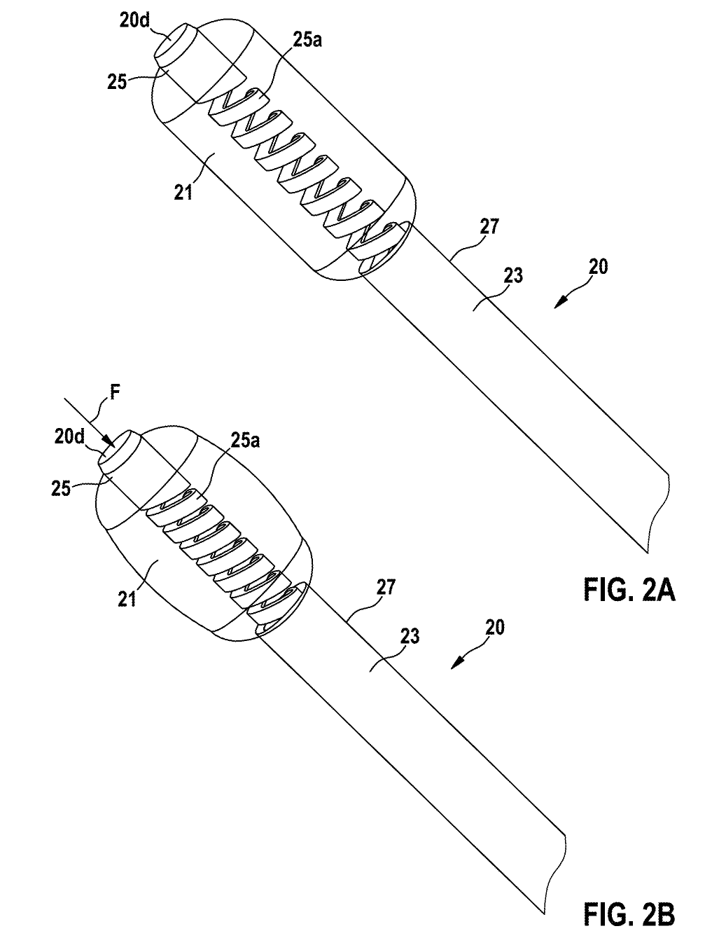

FIGS. 2A-2B show a further catheter according to the present invention, wherein the reference numerals are based on those used in FIGS. 1A-1D, and explanations made above with reference to the first embodiment will not be repeated. FIG. 2A shows a starting state, and FIG. 2B shows an operational state under the effect of a distally acting force “F”.

In this embodiment, a region of inner tube 25 close to the distal end thereof has been converted via an appropriately spirally extending section into a compression spring element 25a which, in conjunction with the elasticity of shape of the sleeve of deformation body 21 and/or the compressible filling thereof, absorbs the distally acting pressure “F” at least to a certain extent. In this case, the distal catheter end 20d is displaced in the proximal direction, even though the entire inner tube 25 need not be displaceable relative to the outer tube 27. The provision of a compression spring region or element can also be combined with displaceability of the entire inner tube 25 relative to the outer tube 27, however. In another form, instead of designing a section of the inner tube 25 as a compression spring element, a separate compression spring element (made of metal, for example) can also be provided between the inner tube 25 and the outer tube 27.

As with the aforementioned embodiment, the displacement in combination with the deformation of the deformation body results in a reduction of the surface pressure on the distal catheter end and, therefore, a reduction of perforation risk.

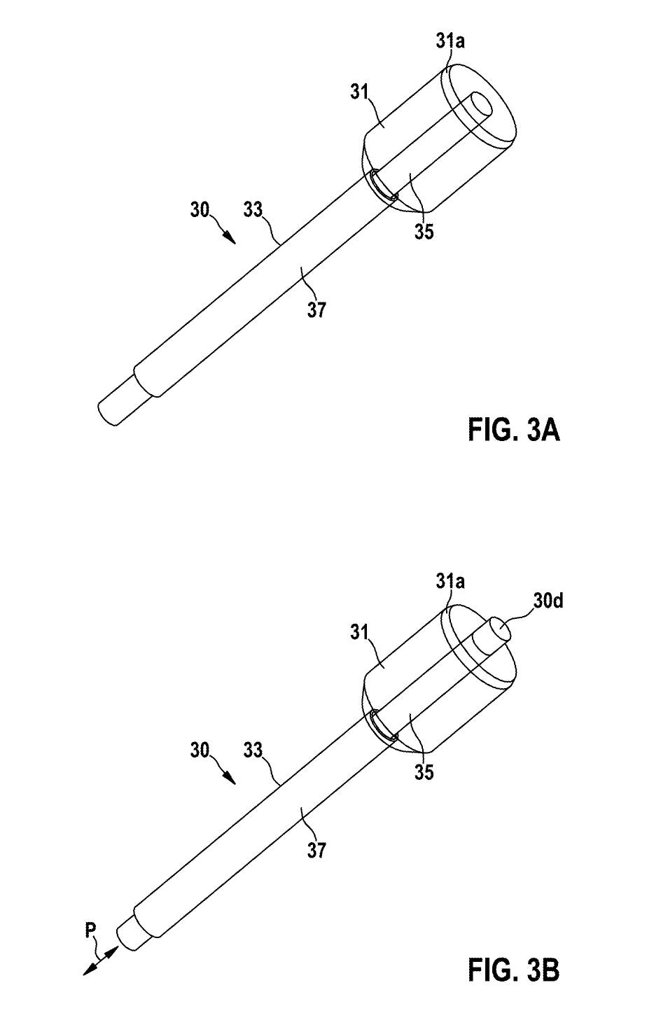

FIGS. 3A-3B show an embodiment having a slightly different function. In this case, the deformation body 31 has a cylindrical shape and comprises a collar 31a on the distal end face, with which the catheter 30 can orient itself upon contact with the vessel wall. As illustrated in FIG. 3B, axial pressure “P” then causes the inner tube 35 to shift slightly over the contact surface defined as a result, and the distal catheter end 30d is thereby pressed slightly into the surrounding body tissue. A desired contact surface and force can be set via the displacement. If an electrode (not depicted) is disposed on the distal end 30d, a desired electrode-tissue contact can be set by way thereof.

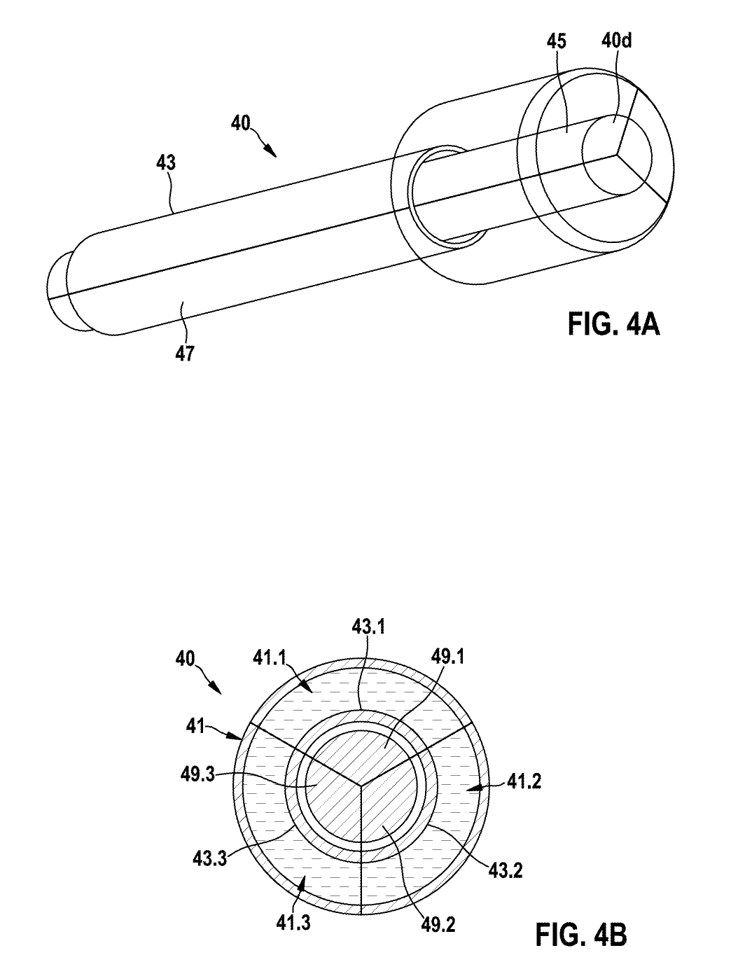

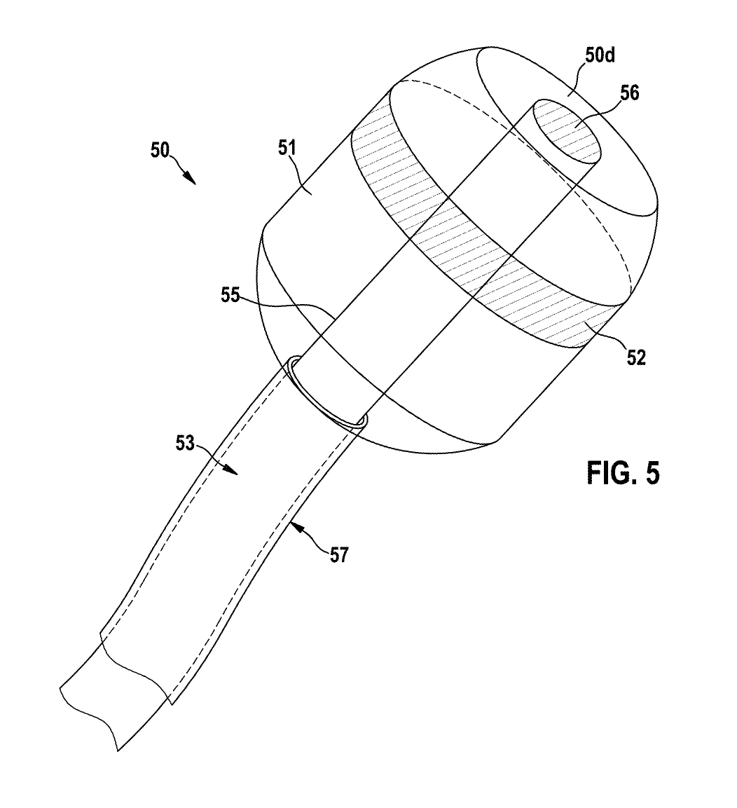

FIGS. 4A-4B present a schematic depiction of a further embodiment of the present invention, specifically a three-chamber catheter 40, in which both the catheter body 43 and the deformation body 41 are subdivided into three chambers. This is shown most clearly in FIG. 4B, where chambers 41.1, 41.2 and 41.3 of the deformation body 41 are labeled individually, as are parts 43.1, 43.2 and 43.3 of the catheter body 43. It is also clear that each chamber has a separate fluid channel 49.1, 49.2 and 49.3 in the interior of the inner tube 45.

If the catheter 40 makes contact with the wall of a vessel or hollow organ during use, this wall contact acts differently on the individual chambers of the deformation body 41, and a specific pressure increase can be tapped at a fluid outlet (not depicted) connected to the particular fluid channel. On the basis thereof, the total force and the direction can be determined, thereby making it possible to obtain additional information about the position of the catheter 40.

If, in one modification, the deformation body 41 is not designed as a three-chamber balloon but, rather, is formed approximately out of three separate foam parts, a similar determination can be made, e.g., via detection of the electrical resistance.

FIG. 5 presents a schematic illustration of the distal end of an electrode catheter 50 according to the present invention comprising a deformation body 51 and a catheter body 53 which has an inner tube 55 and an outer tube 57, and carries two electrodes in the distal region thereof, which can be used, for example, for tissue stimulation and/or sensing tissue potentials. A tip electrode 56 is provided on the distal end 50d of the electrode catheter 50, which can be created, e.g., by a metal coating of the distal end of the inner tube 55. In addition, a tip electrode 52 is provided on the circumference of the deformation body 51, which can be made of an expandable conductive plastic, for example. Alternatively, it is also possible, for example, to provide a meandering metal strip or the like.

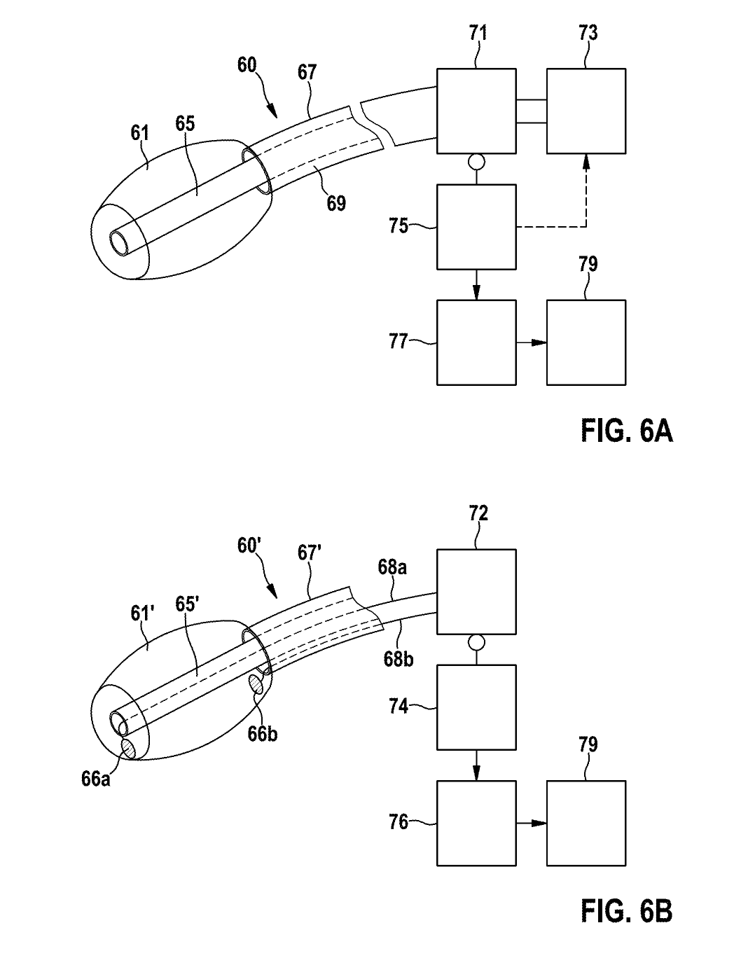

FIG. 6A shows an embodiment of a catheter system comprising a catheter 60 which can be designed according to one of the variants described above, in combination with peripheral devices. A fluid channel 69 is provided between the inner tube 65 and the sleeve of the outer tube 67, via which the interior of the deformation body 61—which is in the form of a fluid-filled balloon—is connected to a fluid connector 71 at the proximal end of the catheter 60. Via fluid connector 71, a fluid source 73 (such as, for example, a carbon dioxide bottle having a controllable valve, or a gas pump) supplies a suitable fluid for filling balloon 61 with a predetermined pressure.

Furthermore, the pressure inside the balloon 61 is measured at fluid connector 71 using pressure sensor 75. The value that is detected is transmitted at the outlet of pressure sensor 75 to an evaluation unit 77 which is connected to a display unit 79. The pressure values that are measured are evaluated and displayed in prepared form to an operator who is handling the catheter 60, thereby informing him of wall contact made by the catheter 60 in the patient's body, and of the related compressive forces.

FIG. 6B shows another catheter system comprising a catheter 60′ which has a foam body 61′ instead of an inflatable balloon as the deformation body. Since a fluid supply for the deformation body is not required in this case, outer tube 67′ comprises a fluid channel, and neither a fluid connector nor a fluid source are provided on the proximal end.

Deformation of deformation body 61′ is detected via two measurement electrode surfaces 66a, 66b in the distal and the proximal region of the deformation body 61′, which are connected via measurement lines 68a and 68b to a measurement power supply 72 comprising an assigned current sensor 74. Situated downstream of the current sensor 74—analogous to the embodiment according to FIG. 6A—are an evaluation unit 76 and, finally, a display unit 79 for providing wall-contact information to the operator. Deformation of deformation body 61′ results in decreased separation between measuring electrodes 66a, 66b and, simultaneously, compression of the foam, which manifests as a change in the resistance in the current path between the measuring electrodes and, therefore, a change in current intensity. The evaluation thereof provides the information required regarding the presence of wall contact, and the intensity thereof.

The embodiments of the present invention are not limited to the above-described examples and emphasized aspects but, rather, are possible in a large number of modifications that lie within the scope of handling by a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. A catheter comprising:
an elongated catheter body which has a distal end and a proximal end in respect of an operating position, wherein the catheter body comprises an inner tube and an outer tube which is displaceable relative to the inner tube: and
a deformation body which has a distal region and a proximal region, wherein the deformation body is disposed on the distal end of the catheter body and wherein the deformation body is connected in the distal region to the distal end of the inner tube, and is connected in the proximal region to the distal end of the outer tube.

2. The catheter according to claim 1, wherein the deformation body comprises a balloon having a fluid filling.

3. The catheter according to claim 2, wherein the balloon is filled with an elastically compressible fluid.

4. The catheter according to claim 2, wherein the balloon is filled with an incompressible fluid.

5. The catheter according to claim 2, wherein the balloon has a fluid connection via a fluid channel in the catheter body to one or more fluid connectors on the proximal end of the catheter.

6. The catheter according to claim 1, wherein the deformation body is made of an elastically compressible material.

7. The catheter according to claim 1, wherein the deformation body comprises a substantially axially acting compression spring device, in particular a metal or plastic spring.

8. The catheter according to claim 7, wherein the substantially axially acting compression spring device comprises a metal or plastic spring.

9. The catheter according to claim 1, wherein the deformation body comprises a flexibly yielding sleeve or jacket layer.

10. The catheter according to claim 1, wherein the deformation body, in an unloaded state, has a diameter that is greater than that of the outer tube, at least in sections.

11. The catheter according to claim 1, wherein the deformation body comprises measuring means, which function electrically or optically, or a measuring connector for detecting a pressure exerted thereupon.

12. The catheter according to claim 1, wherein the deformation body has a multiple-component design and is formed of a plurality of chambers or partial bodies, wherein the chambers or partial bodies have different deformation behavior and/or separate measuring means or measuring connectors for detecting a specific pressure applied thereto.

13. The catheter according to claim 12, wherein each of the chambers has a fluid connection via a fluid channel in the catheter body to a fluid connector on the proximal end of the catheter.

14. The catheter according to claim 12, wherein each of the partial bodies comprise optically or electrically acting measuring means or measuring connections.

15. The catheter according to claim 1, wherein the catheter comprises an electrode lead comprising at least one electrode disposed on the deformation body, or on the distal end of the inner tube and enclosed by the deformation body.

16. The catheter according to claim 15, wherein the at least one electrode is elastically deformable and made of a conductive plastic.

17. A catheter system comprising:
a catheter according to claim 5; and
a measuring device connected to the, or each, proximal fluid connector, or to the, or every other, measuring means or measuring connector for determining a pressure acting on the deformation body or partial regions thereof.
